(19) United States
(12) Patent Application Publication
(10) Pub. No.: US 2026/0177952 A1
(43) Pub. Date: Jun. 25, 2026

(54) TEMPERATURE REGULATING SYSTEM, ABNORMALITY DETERMINING METHOD, AND ABNORMALITY DETERMINING DEVICE

(71) Applicant: OMRON Corporation, Kyoto-shi, Kyoto (JP)

(72) Inventors: Takaaki YAMADA, Kyoto-shi, Kyoto (JP); Yuki KUNIYASU, Kyoto-shi, Kyoto (JP); Hajime TSUBATA, Kyoto-shi, Kyoto (JP); Taichi MIYAMOTO, Kyoto-shi, Kyoto (JP); Masahiro OZAKI, Kyoto-shi, Kyoto (JP); Miho NISHIDE, Kyoto-shi, Kyoto (JP)

(21) Appl. No.: 19/125,380

(22) PCT Filed: Oct. 16, 2023
(86) PCT No.: PCT/JP2023/037433

§ 371 Date: Apr. 29, 2025

(30) Foreign Application Priority Data
Nov. 1, 2022 (JP) 2022-175743

(51) Int. Cl. G03G 15/20; A61B 5/11; G06F 1/20

(52) U.S. Cl. CPC G03G 15/2039; A61B 5/1116; G06F 1/206

(57) ABSTRACT

A temperature regulating system includes a temperature regulator configured to control a heat treatment temperature of a heat treatment apparatus and an abnormality determining device configured to determine an abnormality of the heat treatment apparatus. The abnormality determining device includes a first acquisition unit configured to acquire a feature value of a control waveform of the heat treatment apparatus when the heat treatment temperature is in a settled state and when a disturbance is received and a first determining unit configured to determine that the heat treatment apparatus is abnormal when the feature value acquired by the first acquisition unit exceeds a first threshold value.

### TECHNICAL FIELD

The present disclosure relates to a temperature regulating system, an abnormality determining method, and an abnormality determining device.

### BACKGROUND ART

Patent Document 1 discloses a problem detection system that detects or predicts a problem related to control settling.

### PRIOR ART DOCUMENT

### Patent Document

Patent Document 1: JP-A-2015-69612

### SUMMARY OF THE INVENTION

### Problems to be Solved by the Invention

The problem detection system of Patent Document 1 has room for improvement in accurately determining an abnormality possibility of a heat treatment apparatus.

An object of the present disclosure is to provide a temperature regulating system, an abnormality determining method, and an abnormality determining device capable of more accurately determining an abnormality possibility of a heat treatment apparatus.

### Solutions to the Problems

A temperature regulating system of an aspect of the present disclosure includes:
a temperature regulator configured to control a heat treatment temperature of a heat treatment apparatus; and
an abnormality determining device configured to determine an abnormality of the heat treatment apparatus, wherein
the abnormality determining device includes:
a first acquisition unit configured to acquire a feature value of a control waveform of the heat treatment apparatus when the heat treatment temperature is in a settled state and when a disturbance is received, and
a first determining unit configured to determine that the heat treatment apparatus is abnormal when the feature value acquired by the first acquisition unit exceeds a first threshold value.

“When temperature is settled” refers to a state in which the heat treatment temperature of the heat treatment apparatus remains continuously within a predetermined settled temperature width with the temperature target value as a reference for a certain period of time or more.

The “control waveform” refers to time-series data related to temperature control such as temperature, manipulated variable, and current. The “control waveform” is represented by graphing time-series data related to temperature control with a time axis as a horizontal axis, for example.

The “disturbance” refers to an external action to disturb a state of a control system (for example, temperature or manipulated variable). The “disturbance” includes, for example, a periodic disturbance (a disturbance in a cycle). The periodic disturbance is a “cause of temperature change” that repeatedly occurs as a similar pattern mainly in association with an event of a treatment process of the heat treatment apparatus. The “disturbance” includes “input of a workpiece or a chemical solution”, “opening and closing of a device door”, “target temperature change”, “change in displacement or gas pressure”, and the like.

An abnormality determining method of an aspect of the present disclosure includes:
acquiring a feature value of a control waveform of a heat treatment apparatus when a temperature is in a temperature settled state or when a disturbance is received; and
determining that the heat treatment apparatus is abnormal when the acquired feature value exceeds a first threshold value.

An abnormality determining device of an aspect of the present disclosure includes:
a first acquisition unit configured to acquire a feature value of a control waveform of a heat treatment apparatus when a temperature is in a temperature settling state or when a disturbance is received; and
a determining unit configured to determine that the heat treatment apparatus is abnormal when the feature value acquired by the first acquisition unit exceeds a first threshold value.

### Effects of the Invention

According to the temperature regulating system, the abnormality determining method, and the abnormality determining device of the above aspects, the abnormality possibility of the heat treatment apparatus can be more accurately determined.

### BRIEF DESCRIPTION OF THE DRAWINGS

FIG. 1 is a block diagram illustrating a temperature regulating system according to an embodiment of the present disclosure.

FIG. 2 is a first graph for illustrating an example of a feature value.

FIG. 3 is a second graph for illustrating an example of a feature value.

FIG. 4 is a third graph for illustrating an example of a feature value.

FIG. 5 is a fourth graph for illustrating an example of a feature value.

FIG. 6 is a fifth graph for illustrating an example of a feature value.

FIG. 7 is a sixth graph for illustrating an example of a feature value.

FIG. 8 is a flowchart for illustrating an example of processing of a host controller in an example of abnormality determining processing of the temperature regulating system in FIG. 1.

FIG. 9 is a flowchart for illustrating an example of processing of a temperature regulator in abnormality determining processing of the temperature regulating system in FIG. 1.

FIG. 10 is a flowchart for illustrating an example of threshold setting processing of the temperature regulating system in FIG. 1.

FIG. 11 is a partial block diagram illustrating a first modification of the temperature regulating system in FIG. 1.

FIG. 12 is a flowchart for illustrating an example of slope value sampling processing at the time of temperature rise of the temperature regulating system in FIG. 11.

FIG. 13 is a block diagram illustrating a second modification of the temperature regulating system in FIG. 1.

### DETAILED DESCRIPTION

Hereinafter, an example of the present disclosure will be described with reference to the accompanying drawings. The following description is, essentially, merely illustrative and is not intended to limit the present disclosure, applied products of the present disclosure, or applications of the present disclosure. The drawings are schematic, and ratios and the like of the respective dimensions do not necessarily match those of actual ones.

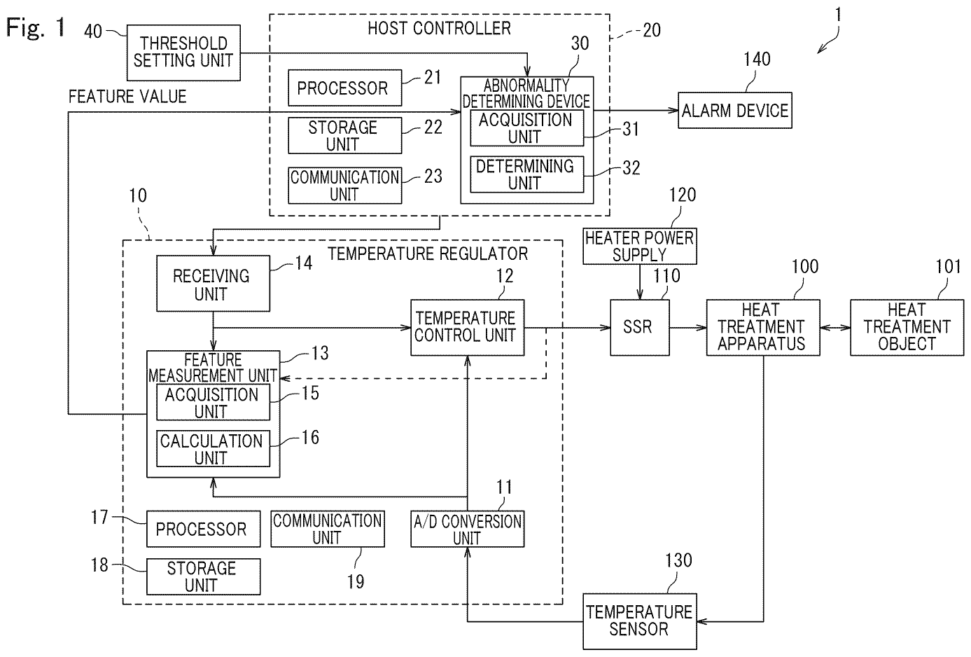

As illustrated in FIG. 1, a temperature regulating system 1 according to an embodiment of the present disclosure includes a temperature regulator 10 and a host controller (an example of a controller) 20 including an abnormality determining device 30. In the present embodiment, the temperature regulating system 1 includes a threshold setting unit 40. The temperature regulator 10 is configured to control a heat treatment temperature of a heat treatment apparatus 100 through a solid state relay (SSR) 110. A heater power supply 120 is connected to the SSR 110. Power is supplied to the heat treatment apparatus 100 through the SSR 110. The heat treatment apparatus 100 is configured to perform heat treatment on a heat treatment object 101 such as a wafer. The heat treatment temperature of the heat treatment apparatus 100 is detected by a temperature sensor 130 and transmitted as an analog signal to the temperature regulator 10.

The temperature regulator 10 includes, for example, a processor 17, a storage unit 18, and a communication unit 19. The processor 17 includes a CPU, an MPU, a GPU, a DSP, an FPGA, an ASIC, or the like. The storage unit 18 includes, for example, an internal recording medium or an external recording medium. The internal recording medium includes a nonvolatile memory and the like. The external recording medium includes a hard disk (HDD), a solid-state drive (SSD), an optical disc device, and the like. The communication unit 19 includes, for example, a communication circuit or a communication module for transmitting and receiving data to and from an external apparatus such as a server.

The temperature regulator 10 includes an A/D conversion unit 11, a temperature control unit 12, a feature measurement unit 13, and a receiving unit 14. The A/D conversion unit 11, the temperature control unit 12, the feature measurement unit 13, and the receiving unit 14 are implemented, for example, by the processor 17 executing a predetermined program stored in the storage unit 18.

Heat treatment temperature data of the heat treatment apparatus 100 detected by the temperature sensor 130 is input to the A/D conversion unit 11 as an analog signal. The A/D conversion unit 11 is configured to convert heat treatment temperature data of the heat treatment apparatus 100 input as an analog signal into a digital signal and to transmit the digital signal to the temperature control unit 12 and the feature measurement unit 13.

The temperature control unit 12 is configured to transmit a control signal (open/close signal) to the SSR 110 based on a target value of the heat treatment temperature of the heat treatment apparatus 100.

The feature measurement unit 13 includes an acquisition unit 15 (an example of a second acquisition unit) and a calculation unit 16 (an example of a first calculation unit).

The acquisition unit 15 is configured to acquire a control waveform. In the present embodiment, the acquisition unit 15 is configured to acquire time-series data of the heat treatment temperature from the A/D conversion unit 11 as the control waveform, and to acquire at least one of the manipulated variable of the control signal, a current, and time-series data of a duty ratio of the control signal from the temperature control unit 12.

The calculation unit 16 is configured to calculate a feature value from the control waveform acquired by the acquisition unit 15. The calculated feature value includes the feature value of the control waveform of the heat treatment apparatus 100 when the heat treatment temperature is in the settled state and when a disturbance is received. The calculated feature value is stored in the storage unit 18 of the temperature regulator 10, for example. In the present embodiment, the calculation unit 16 is configured to sequentially calculate the feature value without waiting for completion of acquisition of the control waveform by the acquisition unit 15.

In the present embodiment, the feature measurement unit 13 is configured to transmit the calculated feature value (for example, only the feature value) to the host controller 20 by wired or wireless communication at and after an acquisition completion of the control waveform by the acquisition unit 15 (in other words, at and after a measurement completion of the control waveform). At this time, the feature measurement unit 13 is configured not to transmit the control waveform acquired by the acquisition unit 15 to the host controller 20 in real time. This is because, for example, in a case where a sampling period is highly frequent (for example, 50 ms), an amount of data of the control waveform becomes larger than the number of sensors and becomes enormous, and a communication load increases. As an example, the feature measurement unit 13 is configured to transmit a current value of the feature value in real-time (in other words, the latest feature value) to the host controller 20 in response to a communication command from the host controller 20. “Not transmitting in real time” includes, for example, periodically transmitting at a cycle longer than the sampling period and transmitting at a predetermined timing such as transmitting at a timing when a data request command from a host is received.

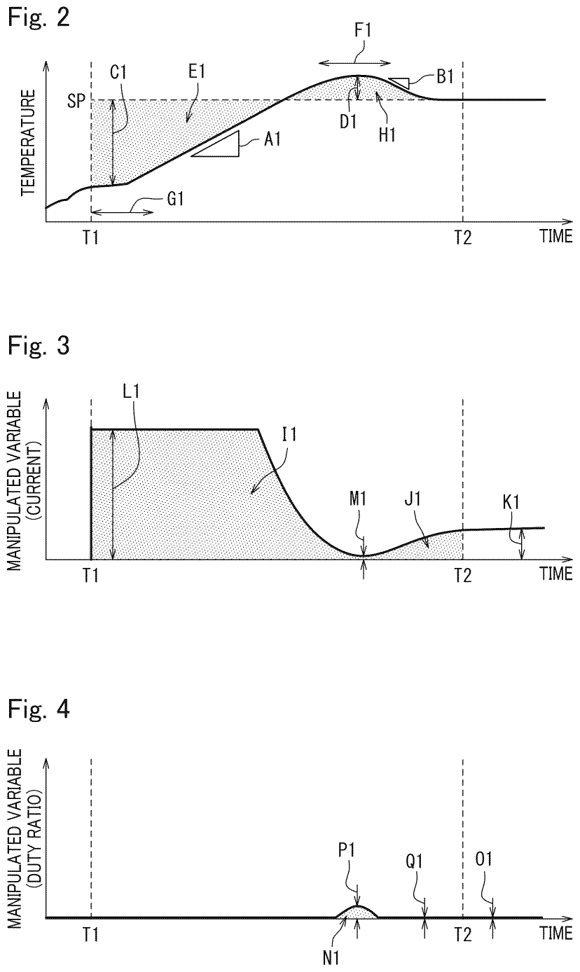

With reference to FIGS. 2 to 4, an example of the feature value that can be calculated from the control waveform acquired at the time of temperature rise will be described below. FIG. 2 illustrates the time-series data of the heat treatment temperature, FIG. 3 illustrates the time-series data of the manipulated variable of the control signal or the current at the time of heating, and FIG. 4 illustrates the time-series data of the manipulated variable of the control signal or the duty ratio of the control signal at the time of cooling. In FIG. 2, the target value of the heat treatment temperature is indicated by “SP”. In FIGS. 2 to 4, feature values are sequentially calculated and updated by the feature measurement unit 13 from a waveform acquisition start time T1. In addition, the latest feature value is transmitted to the abnormality determining device 30 at a point of a waveform acquisition stop time T2.
Maximum slope of temperature waveform: See A1 in FIG. 2
Minimum slope of temperature waveform: See B1 in FIG. 2
Minimum deviation of temperature waveform (undershoot value): See C1 in FIG. 2
Maximum deviation of temperature waveform (overshoot value): See D1 in FIG. 2
Error area from target temperature in temperature waveform: See E1 and H1 in FIG. 2
Overshoot time in temperature waveform: see F1 in FIG. 2
Dead time in temperature waveform: See G1 in FIG. 2
Mean absolute value of deviation in temperature waveform: Area of hatched portion in FIG. 2 (=E1+H1)/waveform acquisition stop time-waveform acquisition start time (=T2−T1)
Steady-state deviation in temperature waveform
Temperature settling time
Average manipulated variable or average current, in manipulated variable waveform (heating): Area of hatched portion in FIG. 3 (=I1+J1)/waveform acquisition stop time-waveform acquisition start time (=T2−T1)
Manipulated variable or current when temperature is settled: See K1 in FIG. 3 (manipulated variable or current when temperature is settled is calculated independently of start and end of waveform measurement)
Maximum manipulated variable or maximum current: See L1 in FIG. 3
Minimum manipulated variable or minimum current: See M1 in FIG. 3
Average manipulated variable or average current, in manipulated variable waveform (cooling): Area of hatched portion in FIG. 4 (=N1)/waveform acquisition stop time-waveform acquisition start time (=T2−T1)
Manipulated variable or current when temperature is settled: See O1 in FIG. 4 (manipulated variable or current when temperature is settled is calculated independently of start and end of waveform measurement)
Maximum manipulated variable or maximum duty ratio: See P1 in FIG. 4
Minimum manipulated variable or minimum duty ratio: See Q1 in FIG. 4
Current standard deviation

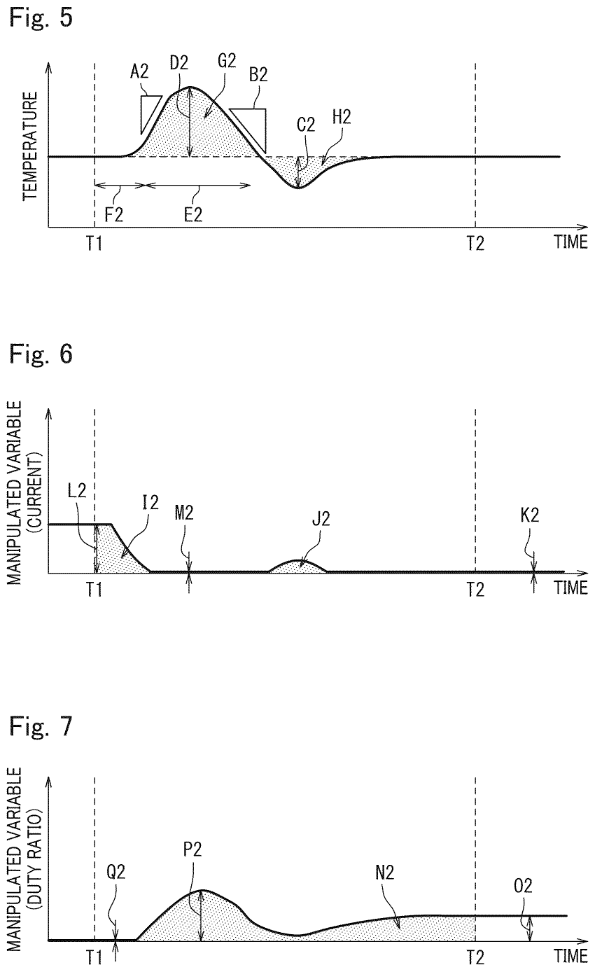

With reference to FIGS. 5 to 7, an example of the feature value that can be calculated from the control waveform acquired at the time of disturbance will be described below. FIG. 5 illustrates the time-series data of the heat treatment temperature, FIG. 6 illustrates the time-series data of the manipulated variable of the control signal or the current at the time of heating, and FIG. 7 illustrates the time-series data of the manipulated variable of the control signal or the duty ratio of the control signal at the time of cooling. In FIG. 5, the target value of the heat treatment temperature is indicated by “SP”. In FIGS. 5 to 7, feature values are sequentially calculated and updated by the feature measurement unit 13 from the waveform acquisition start time T1. In addition, the latest feature value is transmitted to the abnormality determining device 30 at the point of the waveform acquisition stop time T2.
Maximum slope of temperature waveform: See A2 in FIG. 5
Minimum slope of temperature waveform: See B2 in FIG. 5
Minimum deviation of temperature waveform (undershoot amount): See C2 in FIG. 5
Maximum deviation of temperature waveform (overshoot amount): See D2 in FIG. 5
Error area from target temperature in temperature waveform: See G2 and H2 in FIG. 5
Overshoot time in temperature waveform: See E2 in FIG. 5
Dead time in temperature waveform: See F2 in FIG. 5
Mean absolute value of deviation in temperature waveform: Area of hatched portion in FIG. 5 (=G2+H2)/waveform acquisition stop time-waveform acquisition start time (=T2−T1)
Steady-state deviation in temperature waveform
Temperature settling time
Average manipulated variable or average current, in manipulated variable waveform (heating): Area of hatched portion in FIG. 6 (=I2+J2)/waveform acquisition stop time-waveform acquisition start time (=T2−T1)
Manipulated variable or current when temperature is settled: See K2 in FIG. 6 (manipulated variable or current when temperature is settled is calculated independently of start and end of waveform measurement)
Maximum manipulated variable or maximum current: See L2 in FIG. 6
Minimum manipulated variable or minimum current: See M2 in FIG. 6
Average manipulated variable or average current, in manipulated variable waveform (cooling): Area of hatched portion in FIG. 7 (=N2)/waveform acquisition stop time-waveform acquisition start time (=T2−T1)
Manipulated variable or current when temperature is settled: See O2 in FIG. 7 (manipulated variable or current when temperature is settled is calculated independently of start and end of waveform measurement)
Maximum manipulated variable or maximum duty ratio: See P2 in FIG. 7
Minimum manipulated variable or minimum duty ratio: See Q2 in FIG. 7
Current standard deviation

The “duty ratio of control signal” in FIGS. 4 and 7 includes, for example, the duty ratio of the opening/closing signal of the cooling valve. In a case where an element operated by a current (for example, a Peltier element) is used as a cooling means, the feature value can be calculated from the time-series data of “current” instead of the “duty ratio of control signal”.

The receiving unit 14 is configured to acquire a control signal transmitted from the host controller 20. The receiving unit 14 is configured to acquire, for example, the set target value of the heat treatment temperature of the heat treatment apparatus 100 according to the control signal transmitted from the host controller 20. The acquired target value of the heat treatment temperature of the heat treatment apparatus 100 is transmitted to the temperature control unit 12 and the feature measurement unit 13.

The host controller 20 is configured to transmit a control signal to the temperature regulator 10. The host controller 20 includes, for example, a processor 21, a storage unit 22, and a communication unit 23. The processor 21 includes a CPU, an MPU, a GPU, a DSP, an FPGA, an ASIC, or the like. The storage unit 22 includes, for example, an internal recording medium or an external recording medium. The internal recording medium includes a nonvolatile memory and the like. The external recording medium includes a hard disk (HDD), a solid-state drive (SSD), an optical disc device, and the like. The communication unit 23 includes, for example, a communication circuit or a communication module for transmitting and receiving data to and from an external apparatus such as a server.

The abnormality determining device 30 includes an acquisition unit (an example of a first acquisition unit) 31 and a first determining unit 32. The acquisition unit 31 and the first determining unit 32 are implemented, for example, by the processor 21 executing a predetermined program. The predetermined program may be stored in the storage unit 22 of the host controller 20, or a storage unit may be provided in the abnormality determining device 30 and the predetermined program may be stored in the storage unit of the abnormality determining device 30.

The acquisition unit 31 is configured to acquire the feature value calculated by the calculation unit 16 of the feature measurement unit 13. In the present embodiment, the acquisition unit 31 is configured to acquire the first threshold value set by the threshold setting unit 40 in addition to the feature value. The first determining unit 32 is configured to determine that the heat treatment apparatus 100 is abnormal in a case where the feature value acquired by the acquisition unit 31 exceeds the first threshold value acquired by the acquisition unit 31. In a case where it is determined that the heat treatment apparatus 100 is abnormal as a result of the determination by the first determining unit 32, the result is transmitted to an alarm device 140 as an abnormality signal. When the abnormality signal is input, the alarm device 140 transmits an alarm to notify that the heat treatment apparatus 100 is abnormal.

The threshold setting unit 40 is provided in an external apparatus such as a server, for example, and is configured to set a first threshold value used for an abnormality determination of the first determining unit 32. The first threshold value is set, for example, based on the feature value calculated from the heat treatment apparatus 10 in a case of being normal and in a temperature settled state. The threshold setting unit 40 is implemented, for example, by a processor of the external apparatus executing a predetermined program. The threshold setting unit 40 is connected to the host controller 20 in a communicable state. The set first threshold value is transmitted to the abnormality determining device 30 by wired or wireless communication.

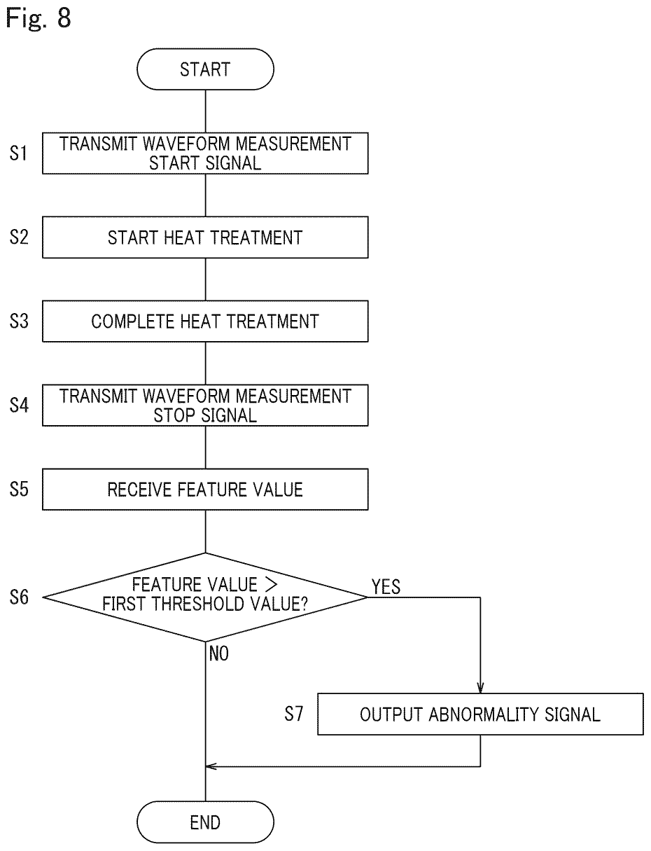

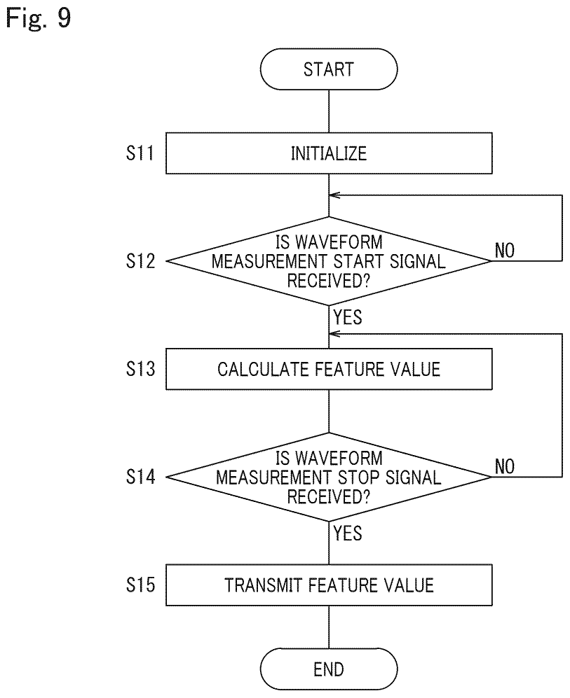

An example of abnormality determining processing of the temperature regulating system 1 will be described with reference to FIGS. 8 and 9. FIG. 8 illustrates an example of processing of the host controller 20 in the abnormality determining processing, and FIG. 9 illustrates an example of processing of the temperature regulator 10. As an example, the abnormality determining processing illustrated in FIGS. 8 and 9 is performed by the processor 21 of the host controller 20 executing a predetermined program stored in the storage unit 22.

As illustrated in FIG. 8, when the abnormality determining processing is started, the host controller 20 transmits a waveform acquisition start signal to the temperature regulator 10 (step S1), and starts the heat treatment in the heat treatment apparatus 100 (step S2).

Thereafter, when the heat treatment in the heat treatment apparatus 100 completes (step S3), the host controller 20 transmits a waveform acquisition stop signal to the temperature regulator 10 (step S4), and the abnormality determining device 30 acquires the feature value calculated by the feature measurement unit 13 (step S5).

When the feature value is acquired, the abnormality determining device 30 determines whether the acquired feature value exceeds the first threshold value (step S6). When it is determined that the acquired feature value exceeds the first threshold value, the abnormality determining device 30 determines that the heat treatment apparatus 100 is abnormal and transmits the abnormality signal (step S7). When the abnormality signal is transmitted, the alarm is transmitted from the alarm device 140, and the abnormality determining processing ends. In a case where it is not determined that the feature value acquired in step S6 exceeds the first threshold value, the abnormality determining processing ends.

As illustrated in FIG. 9, when the abnormality determining processing is started, for example, each unit constituting the temperature regulator 10 is initialized (step S11), and the temperature regulator 10 determines whether a waveform measurement start signal transmitted from the host controller 20 is received (step S12). Step S12 is repeated until it is determined that the waveform measurement start signal is received.

When it is determined that the waveform measurement start signal is received, the temperature regulator 10 acquires the control waveform and calculates the feature value from the acquired control waveform (step S13). Thereafter, the temperature regulator 10 determines whether a waveform measurement stop signal transmitted from the host controller 20 is received (step S14). Step S14 is repeated until it is determined that the waveform measurement stop signal is received.

When it is determined that the waveform measurement stop signal is received, the temperature regulator 10 transmits the calculated feature value to the host controller 20 (step S15), and the abnormality determining processing ends.

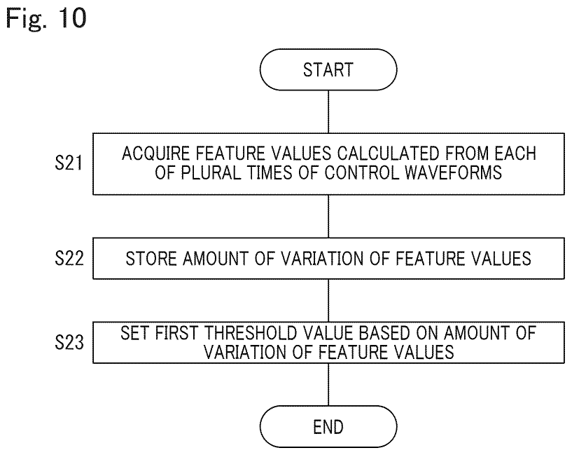

An example of threshold setting processing of the threshold setting unit 40 will be described with reference to FIG. 10. As an example, the threshold setting processing illustrated in FIG. 10 is performed by a processor of an external apparatus executing a predetermined program stored in a storage unit of the external apparatus.

As illustrated in FIG. 10, the threshold setting unit 40 acquires feature values calculated from each of plural times of control waveforms (step S21) and stores the “amount of variation” of the calculated feature values (step S22). As an example, the standard deviation σ of the stored feature values or a difference between the maximum value and the minimum value of the stored feature values is referred to as an “amount of variation”. The “amount of variation” of the calculated feature values is stored in, for example, the storage unit of the external apparatus.

When the “amount of variation” of the calculated feature values is stored, the threshold setting unit 40 sets the first threshold value by, for example, any one of the following methods based on the “amount of variation” of the feature values (step S23), and the threshold setting processing is terminated.

### Reference Waveform Designation Method

A feature value of a control waveform serving as a reference (hereinafter, referred to as a reference waveform) is set as a reference value, and a width of a constant multiple of the “amount of variation” around the reference value is set as a first threshold value. The first threshold value is not limited to a case of having the upper limit value and the lower limit value with respect to the reference value, and may have only the upper limit value or the lower limit value with respect to the reference value. The reference waveform is designated by the user, for example.

### Average Method

An average value of the feature values of plural times of control waveforms is set as a reference value, and a width of a constant multiple of the “amount of variation” around the reference value is set as a first threshold value. This is not limited to the case of having the upper limit value and the lower limit value with respect to the reference value, and may have only the upper limit value or the lower limit value with respect to the reference value.

The temperature regulating system 1 of the present disclosure can exhibit the following advantageous effects.

The temperature regulating system 1 includes a temperature regulator 10 configured to control the heat treatment temperature of the heat treatment apparatus 100, and an abnormality determining device 30 configured to determine an abnormality of the heat treatment apparatus 100. The abnormality determining device 30 includes an acquisition unit 31 configured to acquire the feature value of the control waveform of the heat treatment apparatus 100 when the heat treatment temperature is in the settled state and when a disturbance is received, and a first determining unit 32 configured to determine that the heat treatment apparatus 100 is abnormal in a case where the feature value acquired by the acquisition unit 31 exceeds the first threshold value. With such a configuration, it is possible to implement the temperature regulating system 1 capable of more accurately determining the abnormality possibility of the heat treatment apparatus 100.

For example, in a case of monitoring the variation of the heat treatment temperature waveform, when the heat treatment temperature is feedback-controlled, there is a case where the waveform hardly changes even when the heater capacity or the like varies, and it is difficult to detect an abnormality. In this case, an abnormality of the heat treatment apparatus at the initial stage of operation may not be detected. In the temperature regulating system 1, a change in the feature value of the control waveform (for example, the manipulated variable waveform) of the heat treatment apparatus 100 is monitored. Therefore, even in a case where the heat treatment temperature is feedback-controlled, the abnormality possibility of the heat treatment apparatus 100 can be determined more accurately.

For example, in a case of monitoring the variation of the statistics (for example, average temperature, maximum temperature, and minimum temperature) of the heat treatment temperature, the numerical value serving as the reference is large, and the relative change may be small. In this case, it is difficult to set a threshold value for determining the abnormality of the heat treatment apparatus, and there is a possibility that non-detection and false detection of the abnormality of the heat treatment apparatus easily occur. In addition, when the target value of the heat treatment temperature is changed, it is necessary to reset the threshold value. In the temperature regulating system 1, the variation in the feature value (for example, the feature value related to the target value deviation of the heat treatment temperature) of a control waveform of the heat treatment apparatus 100 is monitored. Therefore, the abnormality possibility of the heat treatment apparatus 100 can be determined more accurately.

For example, the heat treatment apparatus such as a semiconductor manufacturing apparatus and a large continuous furnace always has a high temperature, and the temperature rise timing may be several times or less a year. In a case of monitoring a waveform at the time of temperature rise of such a heat treatment apparatus, the waveform cannot be constantly monitored, and there is a case where the abnormality of the heat treatment apparatus is not detected or falsely detected. In the temperature regulating system 1, the feature value of the control waveform of the heat treatment apparatus 100 at the time of temperature settling and disturbance is monitored. Therefore, even if the heat treatment apparatus 100 is constantly at a high temperature, the abnormality possibility of the heat treatment apparatus 100 can be determined more accurately.

For example, in a case of visually comparing and monitoring the shape of the control waveform of the heat treatment apparatus, the heat treatment apparatus is monitored by human power, and it is difficult to constantly monitor, and variation due to individual differences may occur. In addition, if constant monitoring is to be performed, a large amount of labor cost is required, and there is a possibility that monitoring cost increases. In the temperature regulating system 1, it is possible to determine the abnormality possibility of the heat treatment apparatus 100 from the feature value of the control waveform of the heat treatment apparatus 100 using software, and to constantly automatically monitor the heat treatment apparatus 100 and determine the abnormality possibility of the heat treatment apparatus 100 without relying on human power.

The temperature regulating system 1 may optionally adopt any one or a plurality of configurations out of plural configurations shown next. That is, any one or a plurality of configurations out of the plural configurations shown next may be optionally deleted when included in the embodiment, and may be optionally added when not included in the embodiment. By adopting such a configuration, it is possible to more reliably implement the temperature regulating system 1 capable of more accurately determining the abnormality possibility of the heat treatment apparatus 100.

The temperature regulator 10 includes the acquisition unit 15 configured to acquire the control waveform, and the calculation unit 16 configured to calculate the feature value from the control waveform acquired by the acquisition unit 15.

The abnormality determining device 30 is provided in the host controller 20 configured to control the temperature regulator 10. The temperature regulator 10 is configured to transmit only the calculated feature value to the host controller 20. For example, in a case of sequentially communicating raw waveform data from a temperature regulator to a host controller, data transmission and reception in temperature regulating sampling may increase a communication load and a microprocessor (MPU) load for both the temperature regulator and the host controller. In particular, in a case where the temperature regulator has a large number of channels and simultaneously measures and monitors a large number of channels (for example, 32 channels), the sampling width becomes very coarse, and thus an expensive high-speed communication apparatus may be required to measure an accurate waveform. The temperature regulating system 1, by the above configuration, can measure a more accurate waveform without using an effective high-speed communication apparatus even in a case of simultaneously measuring and monitoring a large number of channels.

The temperature regulator 10 is configured not to transmit the control waveform to the host controller 20 in real time. Accordingly, a communication load between the temperature regulator 10 and the host controller 20 can be reduced.

The calculation unit 16 is configured to sequentially calculate the feature value without waiting for an acquisition completion of the control waveform by the acquisition unit 15. Accordingly, it is not necessary to hold a large capacity of time-series data, and RAM consumption can be reduced.

The temperature regulator 10 is configured to transmit the feature value to the host controller 20 at and after the acquisition completion of the control waveform by the acquisition unit 15. Accordingly, a communication load between the temperature regulator 10 and the host controller 20 can be reduced.

The control waveform is a temperature waveform or a manipulated variable waveform.

The temperature regulating system 1 includes a threshold setting unit 40 configured to set a first threshold value based on an amount of variation of plural times of feature values acquired by the acquisition unit 15.

The amount of variation is a standard deviation of the plural times of feature values or a difference between a maximum value and a minimum value of the plural times of feature values.

The threshold setting unit 40 is configured to set a width that is a constant multiple of the amount of variation as the first threshold value around the feature value of the control waveform serving as a reference.

The threshold setting unit 40 is configured to set a width that is a constant multiple of the amount of variation as the first threshold value around the average value of the plural times of feature values.

The abnormality determining method and the abnormality determining device 30 of the present disclosure can exhibit the following advantageous effects.

The abnormality determining method includes the following steps. With such a configuration, it is possible to more accurately determine the abnormality possibility of the heat treatment apparatus 100.
Acquiring the feature value of the control waveform of the heat treatment apparatus 100 when the temperature is in the temperature settled state or when the disturbance is received.
Determining that the heat treatment apparatus 100 is abnormal when the acquired feature value exceeds the first threshold value.

The abnormality determining device 30 includes the acquisition unit 31 configured to acquire the feature value of the control waveform of the heat treatment apparatus when the temperature is in the temperature settled state or when the disturbance is received, and the first determining unit 32 configured to determine that the heat treatment apparatus 100 is abnormal when the feature value acquired by the acquisition unit 31 exceeds the first threshold value. With such a configuration, it is possible to implement the abnormality determining device 30 capable of more accurately determining the abnormality possibility of the heat treatment apparatus 100.

The temperature regulating system 1 may be configured as follows.

The abnormality determining device 30 is not limited to the case of being provided in the host controller 20. For example, the abnormality determining device 30 may be provided in the temperature regulator 10 or an external apparatus such as a server. In a case where the abnormality determining device 30 is provided in the temperature regulator 10, the determination result on the abnormality possibility of the heat treatment apparatus 100 is transmitted to the host controller 20 in addition to the calculated feature value.

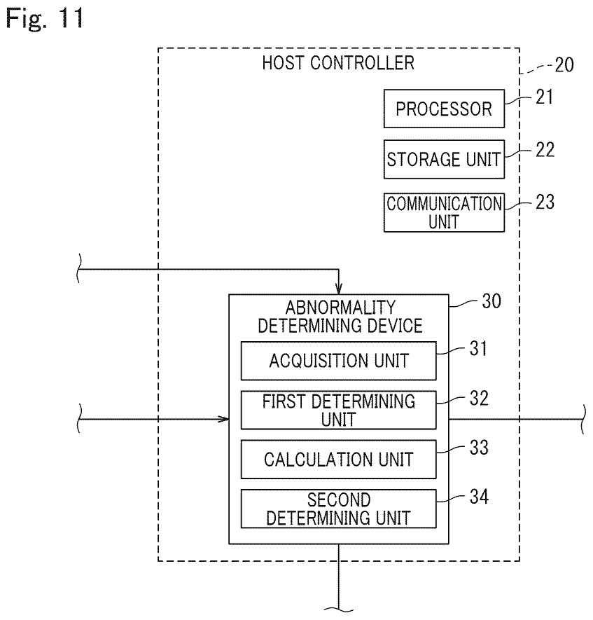

The abnormality determining device 30 may have, for example, the following configuration illustrated in FIG. 11.
A calculation unit (an example of a second calculation unit) 33 configured to calculate the slope of the physical quantity of the heat treatment apparatus 100. The physical quantity is, for example, a temperature, and is detected by a physical quantity sensor (for example, a temperature sensor). The calculation unit 33 is configured to quantize a physical quantity detected by the physical quantity sensor at fixed time intervals to calculate the quantized physical quantity as sampling values. The calculation unit 33 is configured to calculate the slope of the physical quantity of the heat treatment apparatus 100 based on the sampling values on and after which the change width with respect to the sampling value previously calculated becomes the second threshold value or more among calculated sampling values.
A second determining unit 34 configured to determine whether the heat treatment apparatus 100 is abnormal based on the slope value of the physical quantity calculated by the calculation unit 33. For example, the second determining unit 34 determines that the heat treatment apparatus 100 is abnormal when the maximum value of the slope value of the physical quantity becomes equal to or more than the upper limit threshold value or equal to or less than the lower limit threshold value.

For example, when the temperature slope is measured using the temperature value AD-converted for each fixed sampling time width, a resolution of the temperature slope may decrease in a case of a very gentle temperature change. For example, in a case where the sampling width is 1 second, the AD resolution is 0.01° C., and the input temperature slope is 0.001° C./sec, the maximum slope measured value is 0.01° C./sec, and the error is large. As a result, non-detection and false detection of the abnormality of the heat treatment apparatus may easily occur. As a countermeasure, it is conceivable to lengthen the interval of the sampling time, apply a low-pass filter after sampling, and the like. However, in both the countermeasures, unless the parameter is adjusted in real time according to the slope of the input waveform, an appropriate measured value cannot be obtained, and an error may increase in a fixed state, which is not practical.

The abnormality determining device 30 in FIG. 11 determines whether the heat treatment apparatus 100 is abnormal based on the slope value that the maximum value among the slope values calculated by the calculation unit 33 is equal to or greater than the upper limit threshold value or equal to or less than the lower limit threshold value. That is, a minute change in the heat treatment temperature in a range in which the temperature value after quantization does not change is not included in the sampling, and it is possible to more accurately determine the abnormality possibility of the heat treatment apparatus 100. In the abnormality determining device 30 in FIG. 11, sampling is performed at variable time intervals instead of sampling at fixed time intervals.

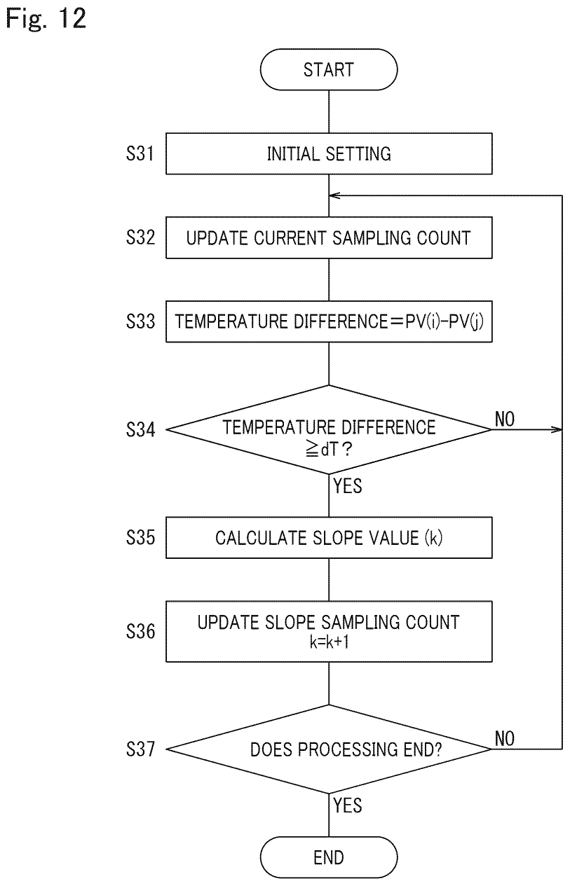

An example of the slope value sampling processing at the time of temperature rise will be described with reference to FIG. 12. As an example, the slope value sampling processing illustrated in FIG. 12 is performed by the processor 21 executing a predetermined program.

As illustrated in FIG. 12, when the slope value calculation processing is started, the calculation unit 33 performs initial setting (step S31). An example of the initial setting will be described below.

$$Temperature sampling with dT = 0.1 ° C .$$

$$Time sampling with dt = 0.05 seconds$$

$$Current sampling count i = 0$$

$$Previous sampling count j = i$$

$$Initial temperature = current temperature PV ( i ) = temperature measured value$$

$$Slope sampling count k = 1$$

When the initial setting is performed, the calculation unit 33 updates the current sampling count (i=i+1) (step S32), and calculates a temperature difference (=PV(i)−PV(j)) between the current temperature PV(i), and the current temperature PV(j) at the previous sampling (step S33). When the temperature difference is calculated, the calculation unit 33 determines whether the calculated temperature difference is equal to or larger than the temperature sampling width dT (step S34). If it is not determined that the calculated temperature difference is equal to or larger than the temperature sampling width dT, the process returns to step S32, and the sampling count is updated.

If it is determined that the calculated temperature difference is equal to or larger than the temperature sampling width dT, the calculation unit 33 updates the previous sampling count and the current temperature PV(i) (j=i, PV(j)=PV(i)), and calculates the slope value (k) (step S35). The slope value (k) is calculated by (temperature measured value at the current sampling-temperature measured value at the previous sampling)/(current sampling time (time taken before step S35=YES at the current sampling)−previous sampling time (time taken before step S35=YES at the current sampling)), that is, the temperature difference/time difference (=dtχ(i−j)) calculated in step S33.

When the slope value (k) is calculated, the calculation unit 33 updates the slope sampling count (k=k+1) (step S36), and determines whether the slope value calculation processing ends (step S37). If it is not determined that the slope value calculation processing ends, the process returns to step S32, and the sampling count is updated.

In the slope value sampling processing in FIG. 12, when the heat treatment temperature decreases from the current temperature PV(i), it is conceivable that the slope value may not be accurately calculated unless the current temperature PV(i) is updated. In order to solve this problem, for example, a step of determining whether the current temperature PV(i) is lower than the current temperature PV(j) at the previous sampling (PV(i)<PV(j)) may be added between step 32 and step S33. In this step, when it is determined that the current temperature PV(i) is lower than the current temperature PV(j) at the previous sampling, the previous sampling count and the current temperature PV(i) are updated (j=i, PV(j)=PV(i)), and the process returns to step S32. When it is not determined that the current temperature PV(i) is lower than the current temperature PV(j) at the previous sampling, the process proceeds to step S33. By adding the above step, the slope value can be calculated more accurately.

The slope value sampling processing illustrated in FIG. 12 can be applied not only when a temperature rises but also when a temperature drops.

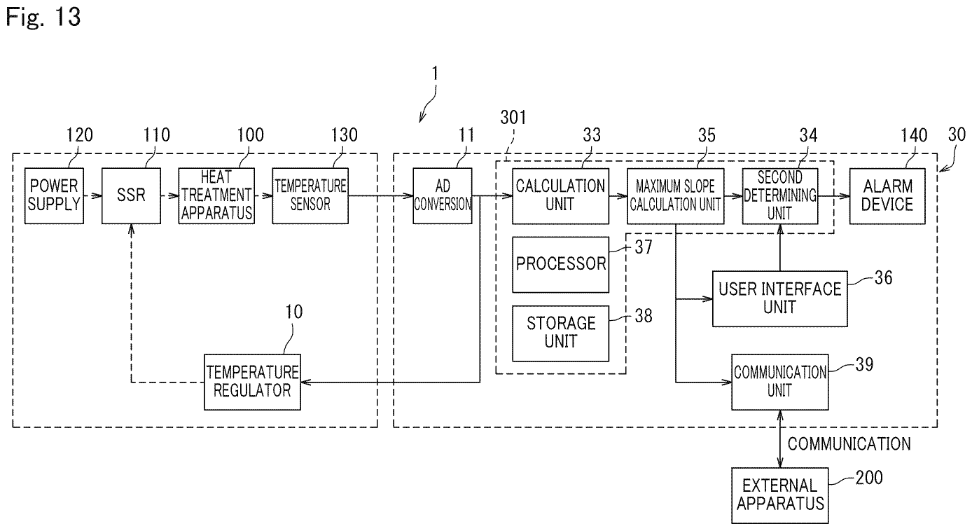

The abnormality determining device 30 in FIG. 11 may be configured as follows.
The abnormality determining device 30 may include a maximum slope calculation unit 35 (see FIG. 13). The maximum slope calculation unit 35 is configured to perform peak hold processing using the slope value of the physical quantity calculated by the calculation unit 33 as an input to calculate a maximum slope value. The peak hold may be processing on not only a positive peak but also a negative peak. In that case, the peak hold is a negative maximum slope.
The calculated slope value and/or maximum slope value may be transmittable to another external apparatus 200 (see FIG. 13) communicatively connected to the abnormality determining device 30.
The abnormality determining device 30 may include a user interface unit 36 (see FIG. 13). For example, the user interface unit 36 may be configured so that the user can change the second threshold value, the upper limit threshold value, and the lower limit threshold value.
The user interface unit 36 may be displayable of the calculated slope value and/or the maximum slope value.
For example, the abnormality determining device 30 may be configured to start a measurement of the physical quantity by a measurement start command and to complete a measurement of the physical quantity by a measurement completion command. The measurement start command and the measurement completion command may be transmittable from the external apparatus 200 to the abnormality determining device 30, for example.

FIG. 13 illustrates an example of the temperature regulating system 1 including the abnormality determining device 30 including the maximum slope calculation unit 35 and the user interface unit 36. In the temperature regulating system 1 in FIG. 13, the abnormality determining device 30 includes the A/D conversion unit 11 and an alarm device 140. The abnormality determining device 30 includes a control device 301 including a processor 37 and a storage unit 38, and a communication unit 39. The calculation unit 33, the maximum slope calculation unit 35, and the second determining unit 34 are implemented, for example, by the processor 37 executing a predetermined program. The processor 37 includes a CPU, an MPU, a GPU, a DSP, an FPGA, an ASIC, or the like. The storage unit 38 includes, for example, an internal recording medium or an external recording medium. The internal recording medium includes a nonvolatile memory and the like. The external recording medium includes a hard disk (HDD), a solid-state drive (SSD), an optical disc device, and the like. The communication unit 39 includes, for example, a communication circuit or a communication module for transmitting and receiving data to and from an external apparatus such as a server.

In the abnormality determining device 30 in FIG. 13, the temperature regulator 10 does not include the acquisition unit 15 and the calculation unit 16. The heat treatment temperature data of the heat treatment apparatus 100 detected by the temperature sensor 130 is transmitted to the calculation unit 33 via the A/D conversion unit 11. The slope value of the heat treatment temperature calculated by the calculation unit 33 is transmitted to the maximum slope calculation unit 35. The maximum slope value calculated by the maximum slope calculation unit 35 is transmitted to the second determining unit 34, the user interface unit 36, and the communication unit 39. The user interface unit 36 transmits the upper limit threshold value and/or the lower limit threshold value to the second determining unit 34.

As described above, the abnormality determining device 30 may include both the first determining unit 32 and the second determining unit 34, or may include only the first determining unit 32 or only the second determining unit 34.

The acquisition unit 15 and the calculation unit 16 of the temperature regulator 10 and the threshold setting unit 40 may be omitted.

The temperature regulator 10 may be configured to transmit the control waveform to the host controller 20 in real time.

The calculation unit 16 of the temperature regulator 10 may be configured to sequentially calculate the feature value with waiting for the acquisition completion of the control waveform by the acquisition unit 15, without being limited to the case of sequentially calculating the feature value without waiting for the acquisition completion of the control waveform by the acquisition unit 15.

The temperature regulator 10 may be configured to transmit the feature value to the host controller 20 before the acquisition completion of the control waveform by the acquisition unit 15, without being limited to the case of transmitting the feature value to the host controller 20 at and after the acquisition completion of the control waveform by the acquisition unit 15.

The first threshold value may be set by another method, without being limited to the case of being set by the processing illustrated in FIG. 10.

The abnormality determining method of the present disclosure can be executed by a computer. That is, the present disclosure includes a program for causing a computer to execute the abnormality determining method and a computer-readable storage medium storing the program for causing a computer to execute the abnormality determining method.

As described above, various embodiments in the present disclosure have been described in detail with reference to the drawings. Lastly, various aspects of the present disclosure will be described. In the following description, as an example, reference numerals are also added.

A temperature regulating system 1 of a first aspect of the present disclosure includes:
a temperature regulator 10 configured to control a heat treatment temperature of a heat treatment apparatus; and
an abnormality determining device 30 configured to determine an abnormality of the heat treatment apparatus, wherein
the abnormality determining device 30 includes:
a first acquisition unit configured to acquire a feature value of a control waveform of the heat treatment apparatus when the heat treatment temperature is in a settled state and when a disturbance is received; and
a first determining unit configured to determine that the heat treatment apparatus is abnormal when the feature value acquired by the first acquisition unit exceeds a first threshold value.

The temperature regulating system 1 of a second aspect of the present disclosure is the temperature regulating system 1 according to the first aspect, wherein
the temperature regulator 10 includes:
a second acquisition unit configured to acquire the control waveform; and
a first calculation unit configured to calculate the feature value from the control waveform acquired by the second acquisition unit.

The temperature regulating system 1 of a third aspect of the present disclosure is the temperature regulating system 1 according to the second aspect, wherein
the abnormality determining device 30 is provided in a controller configured to control the temperature regulator 10, and
the temperature regulator 10 is configured to transmit only the calculated feature value to the controller.

The temperature regulating system 1 of a fourth aspect of the present disclosure is the temperature regulating system 1 according to the third aspect, wherein
the temperature regulator 10 is configured not to transmit the control waveform to the controller in real time.

The temperature regulating system 1 of a fifth aspect of the present disclosure is the temperature regulating system 1 according to any one of the second to fourth aspects, wherein
the first calculation unit is configured to sequentially calculate the feature value without waiting for an acquisition completion of the control waveform by the second acquisition unit.

The temperature regulating system 1 of a sixth aspect of the present disclosure is the temperature regulating system 1 according to any one of the third to fifth aspects, wherein
the temperature regulator 10 is configured to transmit the feature value to the controller at and after an acquisition completion of the control waveform by the second acquisition unit.

The temperature regulating system 1 of a seventh aspect of the present disclosure is the temperature regulating system 1 according to any one of the first to sixth aspects, wherein
the control waveform is a temperature waveform or a manipulated variable waveform.

The temperature regulating system 1 of an eighth aspect of the present disclosure is the temperature regulating system 1 according to any one of the first to seventh aspects, comprising,
a threshold setting unit 40 configured to set the first threshold value based on an amount of variation of plural times of feature values acquired by the first acquisition unit.

The temperature regulating system 1 of a ninth aspect of the present disclosure is the temperature regulating system 1 according to the eighth aspect, wherein
the amount of variation is a standard deviation of the plural times of feature values or a difference between a maximum value and a minimum value of the plural times of feature values.

The temperature regulating system 1 of a tenth aspect of the present disclosure is the temperature regulating system 1 according to the eighth or ninth aspect, wherein
the threshold setting unit 40 is configured to set a width that is a constant multiple of the amount of variation as the first threshold value around the feature value of the control waveform serving as a reference.

The temperature regulating system 1 of an eleventh aspect of the present disclosure is the temperature regulating system 1 according to any one of the eighth to tenth aspects, wherein
the threshold setting unit 40 is configured to set a width that is a constant multiple of the amount of variation as the first threshold value around an average value of the plural times of feature values.

The temperature regulating system 1 of a twelfth aspect of the present disclosure is the temperature regulating system 1 according to any one of the first to eleventh aspects, wherein
the abnormality determining device 30 includes:
a second calculation unit configured to calculate a sampling value by quantizing a physical quantity of the heat treatment apparatus at a fixed time interval, and configured to calculate a slope of the physical quantity based on the sampling value on and after which a change width with respect to the sampling value previously calculated becomes a second threshold value or more among calculated sampling values; and
a second determining unit 34 configured to determine whether the heat treatment apparatus is abnormal based on the slope of the physical quantity calculated by the second calculation unit.

An abnormality determining method of a thirteenth aspect of the present disclosure includes:
acquiring a feature value of a control waveform of a heat treatment apparatus when a temperature is in a temperature settled state or when a disturbance is received; and
determining that the heat treatment apparatus is abnormal when the acquired feature value exceeds a first threshold value.

An abnormality determining device 30 of a fourteenth aspect of the present disclosure includes:
a first acquisition unit configured to acquire a feature value of a control waveform of a heat treatment apparatus when a temperature is in a temperature settling state or when a disturbance is received; and
a first determining unit configured to determine that the heat treatment apparatus is abnormal when the feature value acquired by the first acquisition unit exceeds a first threshold value.

The first to fourteenth aspects may be implemented by a computer program, or may be implemented by any combination of a system, a method, a device, and a computer program.

Appropriately combining any embodiment or modification among the various embodiments or modifications allows the effect of each thereof to be produced. In addition, a combination of embodiments, a combination of examples, or a combination of an embodiment and an example is possible, and a combination of features of different embodiments or examples is also possible.

The present disclosure has been sufficiently described in connection with the preferred embodiments with reference to the accompanying drawings, and various modifications and corrections are apparent for those skilled in the art. It should be understood that as long as such modifications and corrections do not depart from the scope of the present disclosure by the attached claims, they are included therein.

### INDUSTRIAL APPLICABILITY

The temperature regulating system, the abnormality determining method, and the abnormality determining device of the present disclosure can be applied to, for example, abnormality determination of a semiconductor manufacturing apparatus.

### REFERENCE SIGNS LIST

1 temperature regulating system
10 temperature regulator
11 A/D conversion unit
12 temperature control unit
13 feature measurement unit
14 receiving unit
15 acquisition unit
16 calculation unit
17 processor
18 storage unit
19 communication unit
20 host controller
21 processor
22 storage unit
23 communication unit
30 abnormality determining device
31 acquisition unit
32 first determining unit
33 calculation unit
34 second determining unit
35 maximum slope calculation unit
36 user interface unit
37 processor
38 storage unit
39 communication unit
40 threshold setting unit
100 heat treatment apparatus
101 heat treatment object such as wafer
120 heater power supply
130 temperature sensor
140 alarm device
200 external apparatus
301 control device

1. A temperature regulating system, comprising:
a temperature regulator configured to control a heat treatment temperature of a heat treatment apparatus; and
an abnormality determining device configured to determine an abnormality of the heat treatment apparatus, wherein
the abnormality determining device includes:
a first acquisition unit configured to acquire a feature value of a control waveform of the heat treatment apparatus when the heat treatment temperature is in a settled state and when a disturbance is received; and
a first determining unit configured to determine that the heat treatment apparatus is abnormal when the feature value acquired by the first acquisition unit exceeds a first threshold value.

2. The temperature regulating system according to claim 1, wherein
the temperature regulator includes:
a second acquisition unit configured to acquire the control waveform; and
a first calculation unit configured to calculate the feature value from the control waveform acquired by the second acquisition unit.

3. The temperature regulating system according to claim 2, wherein
the abnormality determining device is provided in a controller configured to control the temperature regulator, and
the temperature regulator is configured to transmit only the calculated feature value to the controller.

4. The temperature regulating system according to claim 3, wherein
the temperature regulator is configured not to transmit the control waveform to the controller in real time.

5. The temperature regulating system according to claim 2, wherein
the first calculation unit is configured to sequentially calculate the feature value without waiting for an acquisition completion of the control waveform by the second acquisition unit.

6. The temperature regulating system according to claim 3, wherein
the temperature regulator is configured to transmit the feature value to the controller at and after an acquisition completion of the control waveform by the second acquisition unit.

7. The temperature regulating system 1 according to claim 1, wherein
the control waveform is a temperature waveform or a manipulated variable waveform.

8. The temperature regulating system according to claim 1, comprising,
a threshold setting unit configured to set the first threshold value based on an amount of variation of plural times of feature values acquired by the first acquisition unit.

9. The temperature regulating system according to claim 8, wherein
the amount of variation is a standard deviation of the plural times of feature values or a difference between a maximum value and a minimum value of the plural times of feature values.

10. The temperature regulating system according to claim 8, wherein
the threshold setting unit is configured to set a width that is a constant multiple of the amount of variation as the first threshold value around the feature value of the control waveform serving as a reference.

11. The temperature regulating system according to claim 8, wherein,
the threshold setting unit is configured to set a width that is a constant multiple of the amount of variation as the first threshold value around an average value of the plural times of the feature values.

12. The temperature regulating system according to claim 1, wherein
the abnormality determining device includes:
a second calculation unit configured to calculate a sampling value by quantizing a physical quantity of the heat treatment apparatus at a fixed time interval, and configured to calculate a slope of the physical quantity based on the sampling value on and after which a change width with respect to the sampling value previously calculated becomes a second threshold value or more among calculated sampling values; and
a second determining unit configured to determine whether the heat treatment apparatus is abnormal based on the slope of the physical quantity calculated by the second calculation unit.

13. An abnormality determining method, comprising:
acquiring a feature value of a control waveform of a heat treatment apparatus when a temperature is in a temperature settled state or when a disturbance is received; and
determining that the heat treatment apparatus is abnormal when the acquired feature value exceeds a first threshold value.

14. An abnormality determining device, comprising:
a first acquisition unit configured to acquire a feature value of a control waveform of a heat treatment apparatus when a temperature is in a temperature settling state or when a disturbance is received; and
a first determining unit configured to determine that the heat treatment apparatus is abnormal when the feature value acquired by the first acquisition unit exceeds a first threshold value.
